(19) United States
(12) Patent Application Publication
(10) Pub. No.: US 2010/0021434 A1
(43) Pub. Date: Jan. 28, 2010

(54) Isolated Oligodendrocyte-Like Cells and Populations Comprising Same for the Treatment of CNS Diseases

(75) Inventors: Eldad Melamed, Tel-Aviv (IL); Daniel Offen, Kfar HaRoe (IL); Netta R. Shraga(Blondheim), Doar-Na Galil Tachton (IL)

Correspondence Address: MARTIN D. MOYNIHAN d/b/a PRTSI, INC., P.O. BOX 16446, ARLINGTON, VA, 22215, US

(73) Assignee: Ramot At Tel Aviv University Ltd., Tel-Aviv (IL)

(21) Appl. No.: 12/085,995

(22) PCT Filed: Dec. 7, 2006
(86) PCT No.: PCT/IL2006/001410

§ 371 Date: Sep. 15, 2009

Related U.S. Application Data
(60) Provisional application No. 60/748,219, filed on Dec. 8, 2005.

(51) Int. Cl. A61K 35/12; C12N 5/079

(52) U.S. Cl. 424/93.7; 435/368; 435/377

(57) ABSTRACT

Isolated human cells and populations thereof are provided comprising at least one oligodendrocyte phenotype and at least one mesenchymal stem cell phenotype, wherein the mesenchymal stem cell phenotype is not an oligodendrocyte phenotype. Methods of generating and using same are also provided.

### FIELD AND BACKGROUND OF THE INVENTION

The present invention relates to isolated oligodendrocyte-like cells and populations thereof for the treatment of CNS diseases.

The axons of vertebrate neurons are insulated by a myelin sheath, which greatly increases the rate at which axons can conduct an action potential. Myelin is a cellular sheath formed by special glial cells, namely Schwann cells in the peripheral nervous system and oligodendrocytes in the central nervous system. These glial cells wrap layer upon layer around the axon in a tight spiral, thereby insulating the axonal membrane. However, the sheath is interrupted at regularly spaced nodes of Ranvier, where membrane depolarization can occur. As a result, depolarization of the membrane at one node immediately spreads to the next node. Thus, an action potential propagates along a myelinated axon by jumping from node to node, thereby accelerating transmission of the signal as well as conserving metabolic energy, since the active excitation is confined to the small regions of axonal plasma membrane at the nodes.

The importance of myelination is evidenced by demyelinating diseases such as multiple sclerosis, and demyelinating injuries such as traumatic injuries to the spinal cord. In multiple sclerosis, the myelin sheaths in some regions of the central nervous system are destroyed by an unknown mechanism. When demyelination occurs, the propagation of nerve impulses is significantly slowed, leading to devastating neurological consequences. For example, common symptoms of multiple sclerosis include muscular weakness, slow movements, spasticity, severe fatigue or even disabling exhaustion, visual disturbances, pain, numbness, tingling, urinary dysfunction, sexual dysfunction and mental disturbances.

Current treatments for multiple sclerosis involve slowing down the disease course as well as alleviation of the symptoms or medical complications, rather than addressing the underlying cause of the disease, demyelination. In multiple sclerosis, it appears that cycles of demyelination and remyelination take place, and glial cell transplantation has been investigated as a potential therapy (see, e.g., Smith et al., 2001, Neuroimmunol. 119(2-):261-8.; Brierley et al., 2001, Cell Transplant. 10(3):305-15: Kohama et al., 2001, J. Neurosci. 21(3):944-50). Nevertheless, obtaining large numbers of myelinating cells for transplantation remains a major stumbling block. Glial progenitor cells are available for transplantation; for example, O-2A cells. These give rise in vitro to oligodendrocytes and type II astrocytes. However, although O-2A cells can be grown in culture, only a limited number of divisions are possible [Raff, 1989, Science 243(4897):1450-5]. Moreover, it appears that the O-2A cells that have been injected into animals do not continue to divide, and a large number of cells have to be transplanted. Accordingly, these cells are not suitable for the long term treatment of chronic diseases.

U.S. Pat. No. 5,968,829 teaches culture medium containing CNS neural stem cells that have the capacity to produce neurons, astrocytes, and oligodendrocytes.

PCT publication WO 97/32608 pertains to genetically engineered primary oligodendrocytes for transplantation-mediated delivery in the CNS.

However, it is unclear whether the cells as taught in U.S. Pat. No. 5,968,829 and WO 97/32608 have sufficient replicative capacity to produce the number of cells necessary for human clinical therapy.

U.S. Pat. No. 5,830,621 teaches a human oligodendrocyte cell line deposited with the ATCC under Accession No. CRL 11881. However, the line is essentially free of oligodendrocyte characteristic markers GFAP, GalC, O4, and A2B5.

An alternative source of transplantable cells is pluripotent cells isolated from early embryonic tissue. PCT publication WO 01/88104 describes neural progenitor cell populations obtained by differentiating human ES cells. Populations have been obtained that are over 90% NCAM positive, 35% β-tubulin positive, and 75% A2B5 positive.

However, use of embryonic stem cells is problematic terms of ethics.

The bone marrow contains two major populations of stem cells: hematopoietic and mesenchymal stem cells (MSCs). Characteristics of each population sometimes overlap, but they can be separated by utilizing their unique qualities such as mesenchymal plastic-adherence, or sorting with a specific antigen. Plastic adherent bone marrow MSCs represent a unique population of stem cells capable of differentiation into several types of cells including osteoblasts, adipocytes, chondrocytes and myoblasts. Recent findings indicate that mouse, rat and human bone MSCs can also be induced to differentiate to neuron-like cells [Suzuki et al, Biochem Biophys Res Commun. 2004; 322:918-922; U.S. Pat. Appl. 20050265983].

Studies on transplanted undifferentiated mouse MSCs showed that the cells can migrate into the CNS lesions and differentiate in vivo into the neurons or astrocytes [Lee, et al., Neuropathology, 2003; 23: 169-180].

U.S. Pat. No. 6,989,271 teaches differentiation of MSCs into Schwann cells and not to oligodendrocytes. It is well established that Schwann cells are capable of remyelinating neurons in the CNS. However, Schwann cell remyelination in the CNS does not precisely recapitulate the pattern of remyelination by oligodendroctyes [Kocsis et al., JRRD, Vol. 39, No. 2, P. 287-298]. The density of axonal spacing is less with Schwann cell myelination than with native oligodendrocyte myelination such that it may induce potential negative effects on the system, such as a reduction in axon number.

There remains a widely recognized need for, and it would be highly advantageous to have, an improved source of transplantable cells capable of remyelination devoid of the above limitations.

### SUMMARY OF THE INVENTION

According to one aspect of the present invention there is provided an isolated human cell comprising at least one oligodendrocyte phenotype and at least one mesenchymal stem cell phenotype, wherein the mesenchymal stem cell phenotype is not the oligodendrocyte phenotype.

According to another aspect of the present invention there is provided an isolated human cell comprising at least one mesenchymal stem cell phenotype and at least one oligodendrocyte structural phenotype, wherein the mesenchymal stem cell phenotype is not an oligodendrocyte phenotype.

According to yet another aspect of the present invention there is provided an isolated human cell comprising at least one mesenchymal stem cell phenotype and at least one oligodendrocyte functional phenotype, wherein the mesenchymal stem cell phenotype is not an oligodendrocyte phenotype.

According to still another aspect of the present invention there is provided an isolated cell population comprising human cells wherein:

(i) at least N % of the human cells comprise at least one oligodendrocyte phenotype;

(ii) at least M % of the human cells comprise at least one mesenchymal stem cell phenotype, the mesenchymal stem cell phenotype is not an oligodendrocyte phenotype; and

(iii) at least one of the human cells comprises both the at least one oligodendrocyte phenotype and the at least one mesenchymal stem cell phenotype;

where M and N are each independently selected between 1 and 99.

According to an additional aspect of the present invention there is provided an isolated cell population comprising human cells wherein:

(i) at least N % of the human cells comprise at least one oligodendrocyte structural phenotype;

(ii) at least M % of the human cells comprise at least one mesenchymal stem cell phenotype, the mesenchymal stem cell phenotype is not an oligodendrocyte structural phenotype; and

(iii) at least one of the human cells comprise both the at least one oligodendrocyte structural phenotype and the at least one mesenchymal stem cell phenotype;

where M and N are each independently selected between 1 and 99.

According to yet an additional aspect of the present invention there is provided an isolated cell population comprising human cells wherein:

(i) at least N % of the human cells comprise at least one oligodendrocyte functional phenotype;

(ii) at least M % of the human cells comprise at least one mesenchymal stem cell phenotype, the mesenchymal stem cell phenotype is not an oligodendrocyte functional phenotype; and

(iii) at least one of the human cells comprise both the at least one oligodendrocyte functional phenotype and the at least one mesenchymal stem cell phenotype;

where M and N are each independently selected between 1 and 99.

According to still an additional aspect of the present invention there is provided a method of generating oligodendrocyte-like cells, the method comprising incubating mesenchymal stem cells under conditions sufficient to induce differentiation, thereby generating oligodendrocyte-like cells.

According to a further aspect of the present invention there is provided a method of generating oligodendrocyte-like cells, comprising incubating mesenchymal stem cells in a differentiating medium comprising NT-3, thereby generating oligodendrocyte-like cells.

According to yet a further aspect of the present invention there is provided a method of generating oligodendrocyte-like cells, comprising incubating mesenchymal stem cells in a differentiating medium comprising N2 supplement and bFGF, thereby generating oligodendrocyte-like cells.

According to still a further aspect of the present invention there is provided a method of treating a medical condition of the CNS, the method comprising administering to a subject in need thereof a therapeutically effective amount of the cells or cell populations of the present invention, thereby treating the CNS disease or disorder in the subject.

According to still a further aspect of the present invention there is provided a use of the cells or cell populations of the present invention for the manufacture of a medicament identified for the treatment of a CNS disease or disorder.

According to still a further aspect of the present invention there is provided a cell preparation comprising the cells or cell populations of the present invention.

According to further features in preferred embodiments of the invention described below, the cell preparation further comprises any of the mediums described herein.

According to still a further aspect of the present invention there is provided a pharmaceutical composition comprising as an active agent the cells or cell populations of the present invention and a pharmaceutically acceptable carrier.

According to further features in preferred embodiments of the invention described below, the cells are non-genetically manipulated.

According to still further features in the described preferred embodiments, the at least one oligodendrocyte phenotype is a structural phenotype.

According to still further features in the described preferred embodiments, the at least one oligodendrocyte phenotype is a functional phenotype.

According to still further features in the described preferred embodiments, the cells further comprise an oligodendrocyte functional phenotype.

According to still further features in the described preferred embodiments, the oligodendrocyte functional phenotype is not the mesenchymal stem cell phenotype.

According to still further features in the described preferred embodiments, the cells further comprise an oligodendrocyte structural phenotype.

According to still further features in the described preferred embodiments, the oligodendrocyte structural phenotype is not the mesenchymal stem cell phenotype.

According to still further features in the described preferred embodiments, the oligodendrocyte structural phenotype is a cell size, a cell shape, an organelle size and an organelle number.

According to still further features in the described preferred embodiments, the oligodendrocyte structural phenotype is expression of at least one oligodendrocyte marker.

According to still further features in the described preferred embodiments, the oligodendrocyte marker is a surface marker.

According to still further features in the described preferred embodiments, the oligodendrocyte marker is an internal marker.

According to still further features in the described preferred embodiments, the oligodendrocyte marker is selected from the group consisting of MBP, A2B5 and MOSP.

According to still further features in the described preferred embodiments, the conditions comprise a differentiating medium.

According to still further features in the described preferred embodiments, the differentiating medium comprises NT-3.

According to still further features in the described preferred embodiments, the medium comprises N2 supplement and bFGF.

According to still further features in the described preferred embodiments, a duration of the incubating is about 8 days.

According to still further features in the described preferred embodiments, a concentration of the NT-3 is about 10 ng/ml.

According to still further features in the described preferred embodiments, the differentiating medium further comprises at least one agent selected from the group consisting of Il-1β, N2 supplement, TH, RA, Shh, db-cAMP and forskolin.

According to still further features in the described preferred embodiments, the differentiating medium further comprises N2 supplement and Il-1β.

According to still further features in the described preferred embodiments, the differentiating medium further comprises TH and RA.

According to still further features in the described preferred embodiments, the differentiating medium further comprises Shh, db-cAMP and forskolin.

According to still further features in the described preferred embodiments, the method further comprises culturing the cells in an additional medium prior to the incubating thereby predisposing the cells to differentiate into oligodendrocyte-like cells.

According to still further features in the described preferred embodiments, the additional medium comprises at least one agent selected from the group consisting of PDGF, NT-3, Il-1β, TH, RA and GGF.

According to still further features in the described preferred embodiments, the additional medium comprises PDGF, NT-3 and Il-1β.

According to still further features in the described preferred embodiments, the additional medium comprises TH, RA and GGF.

According to still further features in the described preferred embodiments, the additional medium comprises PDGF and GGF.

According to still further features in the described preferred embodiments, a duration of the incubating is about 5 days.

According to still further features in the described preferred embodiments, a duration of the incubating is about 13 days.

According to still further features in the described preferred embodiments, a concentration of the bFGF is about 10 ng/ml.

According to still further features in the described preferred embodiments, the differentiating medium further comprises at least one agent selected from the group consisting of PDGF, B27 supplement, GGF and db-cAMP.

According to still further features in the described preferred embodiments, the mesenchymal stem cells are obtained by:

(a) culturing a population of cells comprising the mesenchymal stem cells in a proliferating medium capable of maintaining and/or expanding the mesenchymal stem cells; and

(b) selecting the mesenchymal stem cells from the cells resulting from step (a).

According to still further features in the described preferred embodiments, the step (b) is affected by harvesting surface adhering cells.

According to still further features in the described preferred embodiments, the mesenchymal stem cells are bone marrow derived mesenchymal stem cells.

According to still further features in the described preferred embodiments, the mesenchymal stem cells are adipose tissue derived mesenchymal stem cells.

According to still further features in the described preferred embodiments, the cells are autologous cells.

According to still further features in the described preferred embodiments, the cells are non-autologous cells.

According to still further features in the described preferred embodiments, the CNS disease or disorder is multiple sclerosis.

The present invention successfully addresses the shortcomings of the presently known configurations by providing an abundant source of transplantable cells capable of generating myelin.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B are bar graphs illustrating the characterization of MSCs by analysis of surface molecules. Cell surface markers of human (A) and mouse (B) bone marrow-derived mononuclear cell populations (MNCs; dotted columns) were compared with the surface markers of bone marrow derived plastic adherent cell populations cultured in vitro for over 2 weeks (black columns) by flow cytometry. Human bone marrow derived plastic adherent cells showed a significant staining for mesenchymal markers CD29, CD44, and CD105, whereas little or no staining was found for hematopoietic markers CD45, CD19, and CD34. Double staining with CD29 and CD45 or CD29 and CD105, was performed as well, and while few of the MNCs demonstrated mesenchymal double-staining profiles, over 88% of the plastic adherent cells showed a mesenchymal surface marker pattern. Mouse bone marrow derived plastic adherent cells cultured in vitro showed a significant staining for mesenchymal markers CD90 and CD106, whereas no expression of hematopoietic CD45 was detected-compared to high levels of CD45 in the MNCs population. Staining for nonspecific immunoglobulin G (IgG) isotype fluorescence was used as a control. Measurements were made from the cross point of the IgG isotype graph with the specific antibody graph. Quantification is an average±SE of measurements.

FIGS. 2A-F are photomicrographs illustrating the morphology and adypogenic differentiation of human mesenchymal stem cells. Human and mouse bone marrow derived plastic-adherent mesenchymal stem cells display characteristic spindle-like morphology (FIG. 2A) and colony forming units (arrows; human cells FIG. 2B; mouse cells FIG. 2C) in-vitro. Human mesenchymal stem cells cultured in adipogenic differentiation medium for 21 days displayed characteristic morphology and stained positive by Oil red O staining for lipids. Cells in the differentiation medium assumed a round shape (FIG. 2D) with multiple large fat droplets, as detected by Oil red O staining (FIGS. 2E-F). A similar protocol was used for mouse MSCs, and identical results were obtained (not shown). (Magnification FIGS. 2A-D ×100; FIG. 2E ×200; FIG. 2F ×400).

FIGS. 3A-F are photomicrographs illustrating the morphology of mouse MSCs following differentiation experiments in vitro. MSCs from the bone marrow of C57-EGFP transgenic mice were incubated in growth medium supplemented with an assortment of cytokines for 6 days, (FIG. 3A, Il-1β and NT-3; FIG. 3B, NT-3; FIG. 3C, Il-1β; FIG. 3D, RA; FIG. 3E, cAMP; FIG. 3F, control). The cell's morphology did not alter drastically following these procedures. (Magnifications: all ×200).

FIGS. 4A-C are photomicrographs illustrating mouse MSCs acquiring expression of the oligodendrocyte progenitor marker A2B5 following differentiation in-vitro. MSCs from the bone marrow of transgenic C57-EGFP mice (green), were cultured in differentiation mediums composed of standard growth medium supplemented by different cytokines, and then were fixed and stained by antibodies against oligodendrocyte progenitor marker A2B5 (red). NT-3 (50 ng/ml) and IL-1β (20 ng/ml) for 6 days (A), or IL-1β alone (B) or NT-3 alone (C). Cells were photographed by fluorescence-microscope (Magnification: FIG. 4A ×400; FIGS. 4B-C ×200).

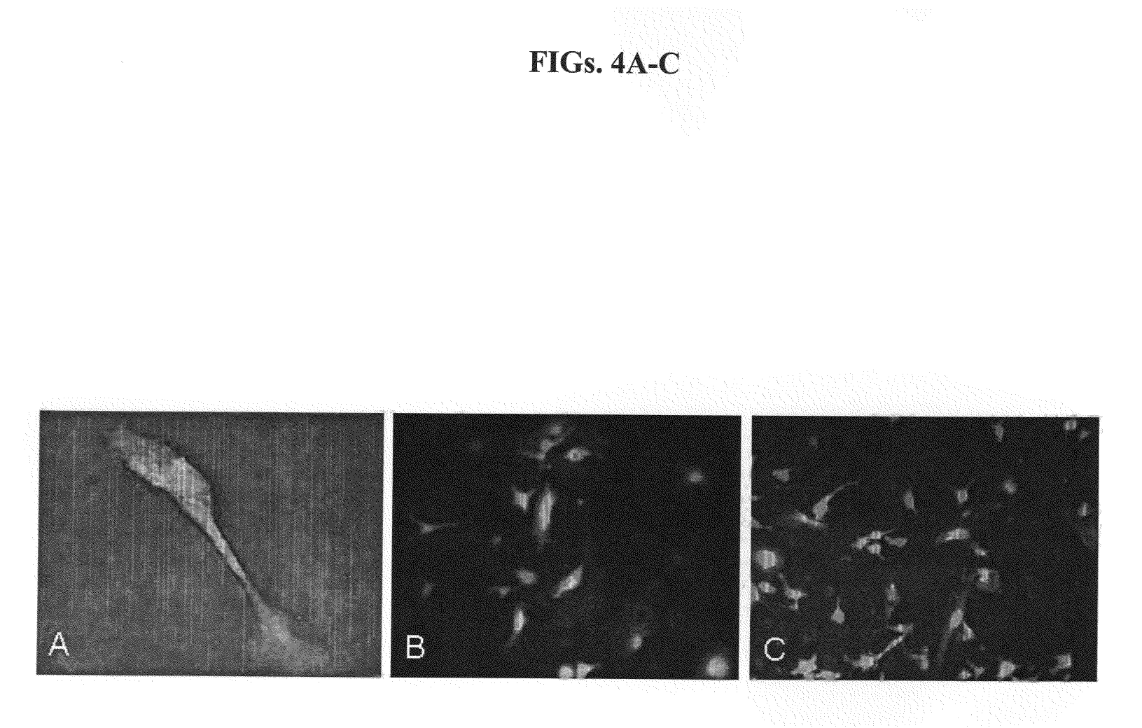

FIGS. 5A-D are graphs and tables illustrating an analysis of mouse MSCs following differentiation protocol in vitro. MSCs from the bone marrow of transgenic EGFP-expressing transgenic mice (A) were cultured in differentiation medium composed of standard growth medium supplemented by IL-1β (20 ng/ml), or in standard growth medium only (control; B) for 4 days. The cells were then stained by antibodies against oligodendrocyte progenitor marker A2B5, and analyzed by flow cytometry (A2B5 staining seen as black lines). Nonspecific staining by second antibody only was used as control (nonspecific staining seen as red lines), and quantitative measurements were made from the cross points of the two lines. Quantitative measurements (D) were made either on the total population (first row), or on the population with very small-sized events omitted (see gated area in A; second row in D).

FIGS. 6A-L are photomicrographs illustrating the morphological changes in human MSCs following differentiation protocols of the present invention. Human MSCs cultured in vitro for over 2 weeks, were incubated in 5 different protocols in an effort to induce oligodendrocyte-like attributes. Initial results on day 5 (FIGS. 6A, 6D, 6G and 6J looked like control cells. By day 8, however, most protocols showed a change in morphology (FIGS. 6B, 6E, 6H and 6K) which by day 12 (experiment end; FIGS. 6C, 6F, 6I and 6L) was characterized by complex cell morphology, more pronounced in some of the protocols, with multiple cell processes. (Magnification: all ×200).

FIG. 7 is a bar graph illustrating the average mRNA levels in human MSCs from two donors, following differentiation protocols A-D, relative to control. Human MSCs from two donors were used to examine the levels of MBP mRNA following induction of oligodendrocyte-like differentiation, by the 5 different protocols. An average of the results of both donors was calculated per treatment (see Table 6 for details). All results are relative to the appropriate control.

FIGS. 8A-G are examples of the morphological complexity of human MSCs following induction of differentiation. Human MSCs incubated in differentiation mediums following protocol D (FIGS. 8A, 8B, 8C, and 8D) and protocol B (FIGS. 8F and 8G) displayed remarkably complex morphology already by day 9 of the differentiation (FIGS. 8A-B), and growing more complex by day 12 (FIGS. 8C, 8D, 8F and 8G) compared to control undifferentiated cells (FIG. 8E). (Magnifications: FIG. 8E ×200, all the rest ×400).

FIGS. 9A-C are photomicrographs illustrating oligodendrocyte progenitor marker, A2B5 expressed by human MSCs following in vitro differentiation protocols. Human MSCs stained positive to early oligodendrocyte progenitor marker A2B5, following differentiation protocols B (FIGS. 9A-B) and D (FIG. 9C). The cells were fixed and stained with anti-A2B5 antibodies (red), and DAPI nuclear staining (blue). (Magnification: FIG. 9A ×100; FIGS. 9B-C ×200).

FIGS. 10A-I are photomicrographs illustrating expression of the oligodendrocyte marker MOSP in human MSCs following in vitro differentiation protocols. Human MSCs stained positive to oligodendrocyte specific marker MOSP, following differentiation protocols D (FIG. 10A-F) and B (FIG. 10G-I). The cells were fixed and stained with anti-A2B5 antibodies (red), and DAPI nuclear staining (blue). (Magnification: FIGS. 10A-B, 10D-E, FIGS. 10G-H ×400; FIGS. 10C, 10F and 10I ×200).

### DESCRIPTION OF THE PREFERRED EMBODIMENTS

The present invention relates to cells and populations thereof which can be transplanted into a patient in order to treat a CNS disease or disorder such as multiple sclerosis.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The importance of myelination is demonstrated by the demyelinating disease multiple sclerosis, in which myelin sheaths in some regions of the central nervous system are destroyed by an unknown mechanism. The significance of myelination is also demonstrated in many other neurodegenerative disease, in which myelinated neurons are injured. Where this happens, the propagation of nerve impulses is greatly slowed, often with devastating neurological consequences.

Restoration of myelin has been proposed as a treatment therapy in order to address the underlying cause of such diseases. However, obtaining large numbers of myelinating cells for transplantation remains a major stumbling block.

Whilst reducing the present invention to practice, the present inventors have found culturing conditions under which mesenchymal stem cells (MSCs) may be differentiated into cells having an oligodendrocyte phenotype. Accordingly, the present inventors propose that such differentiated MSCs can be used to treat patients with CNS diseases or disorders following transplantation.

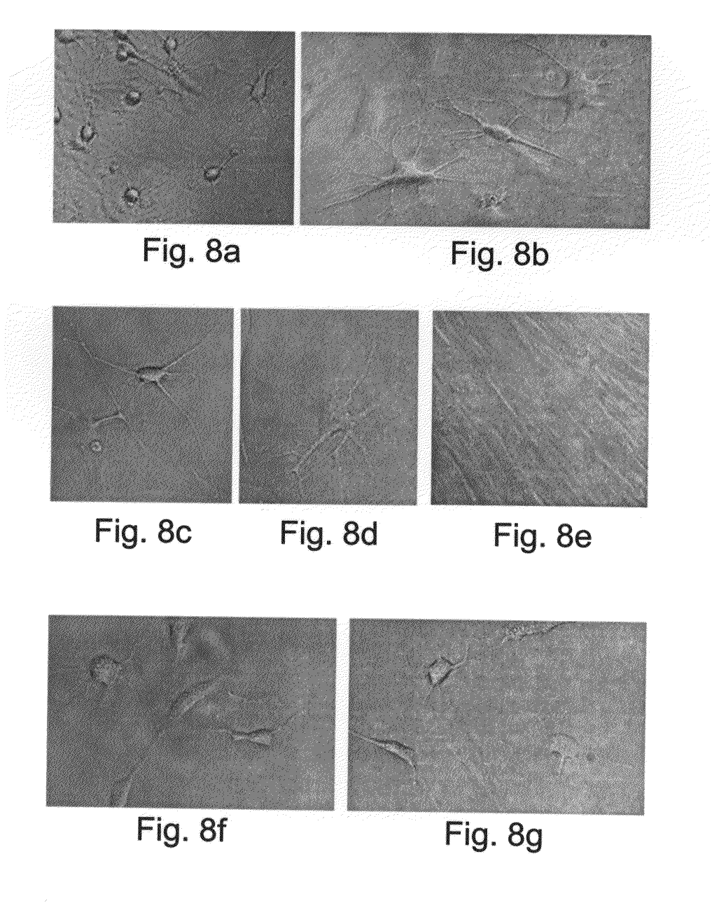

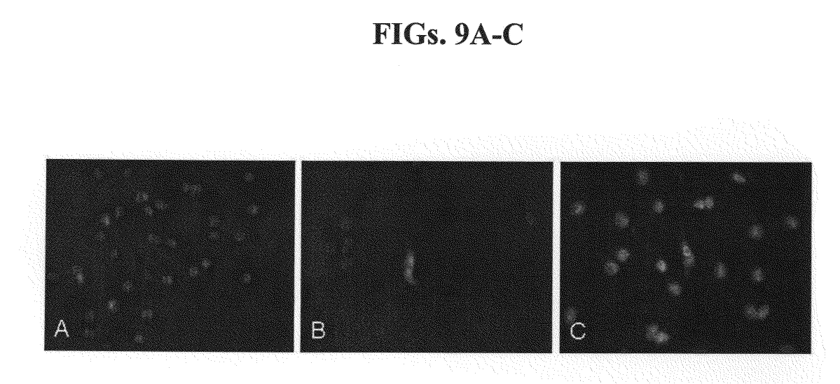

The present inventors have shown that human MSCs differentiated according to several novel two-step protocols represent an oligodendrocyte like morphology (FIGS. 8A-G) accompanied by the presence of oligodendrocyte specific markers (FIGS. 9A-C and 10A-I).

Thus, according to one aspect of the present invention there is provided a method of generating oligodendrocyte-like cells comprising incubating mesenchymal stem cells under conditions sufficient to induce differentiation.

As used herein, the phrase “oligodendrocyte-like cells” refers to cells comprising at least one oligodendrocytic phenotype which allows same to mediate an oligodendrocyte activity, i.e., generate myelin. Accordingly, the oligodendrocyte-like cells of the present invention may comprise phenotypes of an oligodendrocyte precursor cell (OPC) or a mature well-differentiated oligodendrocyte.

Such phenotypes are further described hereinbelow.

As used herein, the term “differentiating” refers to changing, either partially, or completely the phenotype of the mesenchymal stem cell into a cell which comprises either a partial or total phenotype of an oligodendrocyte.

The term “mesenchymal stem cell” or “MSC” refers to fetal or postnatal (e.g., adult) cells which irreversibly differentiate (either terminally or non-terminally) to give rise to cells of a mesenchymal cell lineage and which are also capable of dividing to yield stem cells. The mesenchymal stem cells of the present invention may be of a syngeneic or allogeneic source, although the first is preferred.

According to a preferred embodiment of this aspect of the present invention the mesenchymal stem cells are not genetically manipulated (i.e. transformed with an expression construct) to generate the cells and cell populations described herein.

It will be appreciated that the cells of the present invention may be derived from any stem cell, although preferably not ES cells.

Mesenchymal stem cells may be isolated from various tissues including but not limited to bone marrow, peripheral blood, blood, placenta and adipose tissue. A method of isolating mesenchymal stem cells from peripheral blood is described by Kassis et al [Bone Marrow Transplant. 2006 May; 37(10):967-76]. A method of isolating mesenchymal stem cells from placental tissue is described by Zhang et al [Chinese Medical Journal, 2004, 117 (6):882-887]. Methods of isolating and culturing adipose tissue, placental and cord blood mesenchymal stem cells are described by Kern et al [Stem Cells, 2006; 24:1294-1301].

According to a preferred embodiment of this aspect of the present invention, the mesenchymal stem cells are human.

Bone marrow can be isolated from the iliac crest of an individual by aspiration. Low-density BM mononuclear cells (BMMNC) may be separated by a FICOL-PAGUE density gradient. In order to obtain mesenchymal stem cells, a cell population comprising the mesenchymal stem cells (e.g. BMMNC) may be cultured in a proliferating medium capable of maintaining and/or expanding the cells. According to one embodiment the populations are plated on polystyrene plastic surfaces (e.g. in a flask) and mesenchymal stem cells are isolated by removing non-adherent cells. Alternatively mesenchymal stem cell may be isolated by FACS using mesenchymal stem cell markers.

Preferably the MSCs are at least 50% purified, more preferably at least 75% purified and even more preferably at least 90% purified.

Following isolation the cells are typically expanded by culturing in a proliferation medium capable of maintaining and/or expanding the isolated cells ex vivo as described in Example 1 hereinbelow. The proliferation medium may be DMEM, alpha-MEM or DMEM/F12. Preferably, the proliferation medium is DMEM. Preferably, the proliferation medium further comprises SPN, L-glutamine and a serum (such as fetal calf serum or horse serum) such as described in the General Materials and Methods of the Examples section which follows.

Differentiation to oligodendrocyte-like cells can be effected by incubating the MSCs in differentiating media such as those described in U.S. Pat. No. 6,528,245 and by Sanchez-Ramos et al. (2000); Woodburry et al. (2000); Woodburry et al. (J. Neurisci. Res. 96:908-917, 2001); Black and Woodbury (Blood Cells Mol. Dis. 27:632-635, 2001); Deng et al. (2001), Kohyama et al. (2001), Reyes and Verfatile (Ann. N.Y. Acad. Sci. 938:231-235, 2001) and Jiang et al. (Nature 418:47-49, 2002).

The differentiating media may be DMEM or DMEM/F12, or any other medium that supports neuronal growth. According to a preferred embodiment of this aspect of the present invention, the medium is Neurobasal medium (e.g. Cat. No. 21103049, Invitrogen, Ca, U.S.A.).

Preferably, the MSCs are differentiated for a period of time between about 5 days to about 13 days in the differentiating medium so that differentiation into oligodendrocyte-like cells may occur. The exact number of days is dependent upon the particular differentiating agents added to the medium and may be determined empirically.

According to one embodiment of this aspect of the present invention, the cells are incubated (e.g. for about 8 days) in a differentiating medium comprising NT-3 (e.g. 10 ng/ml).

As used herein, “NT-3” refers to a human polypeptide, or mammalian homologues thereof, having a protein sequence essentially as published at Jones et al., Proc. Natl. Acad. Sci. (USA) 87: 8060-8064 (1990); Maisonpierre et al., Genomics 10: 558-568 (1991); Kaisho et al., FEBS Lett. 266: 187-191 (1990); WO 91/03569; and set forth in GenBank Accession No. M37763. NT-3 is commercially available e.g. PeproTech (www.peprotech.com).

According to this embodiment, the differentiating medium typically comprises other differentiating agents including, but not limited to Il-1β, N2 supplement, TH, RA, Shh, db-cAMP and forskolin.

Thus, according to one embodiment of this aspect of the present invention, the differentiating medium comprises NT-3, N2 supplement and Il-1β (e.g. 20 ng/ml), also referred to herein as differentiating medium B.

According to another embodiment of this aspect of the present invention, the differentiating medium comprises NT-3, TH (e.g. 30 ng/ml) and RA (1 μM), also referred to herein as differentiating medium C.

According to yet another embodiment of this aspect of the present invention, the differentiating medium comprises NT-3, Shh (e.g. 300 ng/ml), db-cAMP (e.g. 1 nM) and forskolin (e.g. 5 μM), also referred to herein as differentiating medium D.

Mesenchymal stem cells may be incubated in an “additional medium” for at least 3 days, preferably 5 days, prior to their incubation in the differentiation mediums of the present invention in order to predispose the cells to differentiate into oligodendrocyte-like cells.

The “additional medium” according to this aspect of the present invention may comprises differentiating agents such as PDGF, NT-3, Il-1β, TH, RA and GGF.

According to one embodiment of this aspect of the present invention, the additional medium comprises PDGF (e.g. 20 ng/ml), NT-3 (e.g. 10 ng/ml) and Il-1β (20 ng/ml), also referred to herein as additional medium B.

According to another embodiment of this aspect of the present invention, the additional medium comprises TH (e.g. 30 ng/ml), RA (e.g. 1 μM) and GGF (50 ng/ml), also referred to herein as additional medium C.

According to yet another embodiment of this aspect of the present invention, the additional medium comprises PDGF (e.g. 20 ng/ml) and GGF (e.g. 50 ng/ml), also referred to herein as additional medium D.

Any combination of additional medium and differentiating medium is envisaged by the present invention, although particularly preferred is a combination of additional medium C with differentiating medium C, additional medium D with differentiating medium D and additional medium E with differentiating medium E.

According to yet another embodiment of this aspect of the present invention, the mesenchymal stem cells are incubated in a differentiating medium comprising N2 supplement and bFGF in order to generate the oligodendrocyte cells of the present invention.

As used herein, “N2 supplement” refers to a mixture of components comprising about 5 μg/ml insulin; 20 nM progesterone; 100 μM putrescine; 30 nM selenium; and 100 μg/ml transferrin. N2 supplement is wildely available from such Companies as e.g. Sigma Aldrich and Invitrogen, Carlsbad, Calif.

The term “bFGF” refers to a polypeptide which is also commonly known as basic fibroblast growth factor or FGF. It is a member of the fibroblast growth factor. bFGF is commercially available from R&D (www.rndsystems.com). According to an embodiment of this aspect of the present invention, the concentration of FGF is about 10 ng/ml.

The differentiating medium of this aspect of the present invention may comprise other differentiating agents including, but not limited to PDGF, B27 supplement, GGF and db-cAMP.

The differentiating media (including the additional differentiating medium may also comprise other agents such as neurotrophic factors (e.g. BDNF, CNTF, GDNF, NTN, NT3 or LIF), hormones, growth factors (e.g. TGF-β3, TGF-α, and FGF-8), vitamins, hormones e.g., insulin, progesterone and other factors such as sonic hedgehog, bone morphogenetic proteins, forskolin, retinoic acid, ascorbic acid, putrescin, selenium and transferrin.

Cell populations obtained according to the methods describe herein are typically non-homogeneous.

Thus, according to another aspect of the present invention there is provided an isolated population of human cells wherein:

(i) at least N % of the cells comprise at least one oligodendrocyte phenotype;

(ii) at least M % of the cells comprise at least one mesenchymal stem cell phenotype, the mesenchymal stem cell phenotype is not an oligodendrocyte phenotype; and (iii) at least one of the human cells comprises both the at least one oligodendrocyte phenotype and the at least one mesenchymal stem cell phenotype; where M and N are each independently selected between 1 and 99.

The term “isolated” as used herein refers to a population of cells that has been removed from its in-vivo location (e.g. bone marrow, neural tissue). Preferably the isolated cell population is substantially free from other substances (e.g., other cells) that are present in its in-vivo location.

As used herein, the phrase “oligodendrocyte phenotype” refers to a structural and/or functional parameter typical (e.g. unique) to an oligodendrocyte which may be used to distinguish between the differentiated MSCs of the present invention and non-differentiated MSCs. The oligodendrocyte phenotype may comprise a single or a number of features which may be used to distinguish between the differentiated MSCs of the present invention and non-differentiated MSCs.

It will be appreciated that the functional parameters may overlap with the structural parameter e.g., expression of myelin markers.

Preferably the functional oligodendrocyte phenotype comprises the ability to generate myelin on nerve cells.

Examples of mature oligodendrocyte functional phenotypes include, expression of at least one oligodendrocyte marker.

As used herein the phrase “oligodendrocyte marker” refers to a polypeptide which is either selectively or non-selectively expressed in an oligodendrocyte. Preferably, the marker has a significantly (e.g. at least 10 fold) higher expression in oligodendrocytes as opposed to other cells, such as Schwann cells and naïve mesenchymal stem cells. The oligodendrocyte marker may be expressed on the cell surface or internally.

Examples of mature oligodendrocyte markers include, but are not limited to proteolipid protein (PLP), MBP, myelin-associated glycoprotein (MAG), myelin oligodendrocyte glycoprotein (MOG), in addition to galactocerebrosides (O1, GalC).

According to a preferred embodiment of this aspect of the present invention, the oligodendrocyte-like cells express MBP and/or MOG.

Examples of OPC functional phenotypes include, but are not limited to, mitotic (i.e. that can divide and be expanded for three or more passages in culture) and migratory capacities as well as the potential to differentiate into a myelinating phenotype to effect myelination in vivo and in vitro.

Examples of OPC marker expression include, but are not limited to, PDGF-receptor, O4 sulfatide marker, Nkx2.2, Sox10, Olig1/2, oligodendrocyte specific protein (OSP), 2′,3′-cyclic nucleotide-3′-phosphodiesterase (CNP), adenomatous polyposis coli (APC); NG2 (Chondroitin sulfate proteoglycan), A2B5, GD3 (ganglioside), nestin, vimentin and E- or PSA-NCAM.

As mentioned hereinabove a percentage of the cells of the cell populations of the present invention may additionally or alternatively comprise a structural oligodendrocyte phenotype.

Examples of structural oligodendrocyte phenotypes include a cell size, a cell shape, an organelle size and an organelle number. Thus, mature oligodendrocyte structural phenotypes include, a branched and ramified phenotype and formation of myelin membranes (See FIGS. 8A-G). Examples of OPC structural phenotype include, but are not limited to elongated, bipolar or multipolar morphology. For example only OPCs, but not mature oligodendrocytes, incorporate bromodeoxyuridine (BUdR), a hallmark of mitosis.

These structural phenotypes may be analyzed using microscopic techniques (e.g. scanning electro microscopy). Antibodies or dyes may be used to highlight distinguishing features in order to aid in the analysis.

As mentioned hereinabove, a percentage of cells of the cell populations comprise at least one mesenchymal stem cell phenotype which is not present in typical oligodendrocyte cells. Such stem cell phenotypes are typically structural. For example, the cells of the present invention may show a morphology similar to that of mesenchymal stem cells (a spindle-like morphology). Alternatively or additionally the cells of the present invention may express a marker (e.g. surface marker) typical to mesenchymal stem cells but atypical to native oligodendrocyte cells. Examples of mesenchymal stem cell surface markers include but are not limited to CD105+, CD29+, CD44+, CD90+, CD34−, CD45−, CD19−, CD5−, CD20−, CD11B− and FMC7−. Other mesenchymal stem cell markers include but are not limited to tyrosine hydroxylase, nestin and H-NF.

The cell populations of the present invention also include cells which display both an oligodendrocyte phenotype and a mesenchymal stem cell phenotype. The mesenchymal stem cell phenotype is preferably not an oligodendrocyte phenotype.

Preferably, when cells comprise both the oligodendrocyte and mesenchymal stem cell phenotypes described hereinabove, their oligodendrocyte phenotype is unique to oligodendrocytes e.g. myelination of nerve cells in a particular pattern distinct from that obtained with Schwann cells and/or expression of MOSP. The cells may comprise a single oligodendrocyte phenotype unique to oligodendrocyte (e.g. expression of a selectively expressed marker) or a combination of non-unique oligodendrocyte phenotypes which in combination represent a phenotype unique to oligodendrocytes.

According to one embodiment of the present invention, the oligodendrocyte phenotype of any of the cells of the populations of the present invention is as close as possible to native oligodendrocytes.

The percentage of cells which comprise an oligodendrocyte phenotype may be raised or lowered according to the intended needs. Thus for example, the cell populations may be enriched for oligodendrocytes by FACS using an antibody specific for an oligodendrocyte cell marker. Examples of such oligodendrocyte markers are described hereinabove. If the cell marker is an internal marker, preferably the FACS analysis comprises antibodies or fragments thereof which may easily penetrate a cell and may easily be washed out of the cell following detection. The FACS process may be repeated a number of times using the same or different markers depending on the degree of enrichment and the cell phenotype required as the end product.

M % may be any percent from 1% to 99% e.g. 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%.

N % may be any percent from 1% to 99% e.g. 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%.

According to another embodiment of this aspect of the present invention the cell populations may be enriched for cells comprising both an oligodendrocyte phenotype and a mesenchymal stem cell phenotype such that a homogeneous population of cells are generated.

Thus, according to yet a further aspect of the present invention there is provided an isolated human cell comprising at least one oligodendrocyte phenotype and at least one mesenchymal stem cell phenotype, wherein the mesenchymal stem cell phenotype is not an oligodendrocyte phenotype.

Once differentiated and optionally isolated, the cells may be tested (in culture) for their oligodendrocyte phenotype (e.g. ability to generate myelin). The cultures may be comparatively analyzed for an oligodendrocyte phenotype, using biochemical analytical methods such as immunoassays, Western blot and Real-time PCR as described in Examples 3 of the Examples section which follows, or by enzyme activity bioassays.

The cells and cell populations of the present invention may be useful for a variety of therapeutic purposes. Diseases and conditions of the nervous system that result from the deterioration of, or damage to, the myelin sheathing generated by myelin producing cells are numerous. Myelin may be lost as a primary event due to direct damage to the myelin or as a secondary event as a result of damage to axons and neurons. Primary events include neurodegenerative diseases such as multiple sclerosis (MS), human immunodeficiency MS-associated myelopathy, transverse myelopathy/myelitis, progressive multi focal leukoencepholopathy, central pontine myelinolysis and lesions to the myelin sheathing (as described below for secondary events). Secondary events include a great variety of lesions to the axons or neurons caused by physical injury in the brain or spinal cord, ischemia diseases, malignant diseases, infectious diseases (such has HIV, Lyme disease, tuberculosis, syphilis, or herpes), degenerative diseases (such as Parkinson's, Alzheimer's, Huntington's, ALS, optic neuritis, postinfectious encephalomyelitis, adrenoleukodystrophy and adrenomyeloneuropathy), schizophrenia, nutritional diseases/disorders (such as folic acid and Vitamin B12 deficiency, Wemicke disease), systemic diseases (such as diabetes, systemic lupus erthematosis, carcinoma), and toxic substances (such as alcohol, lead, ethidium bromide); and iatrogenic processes such as drug interactions, radiation treatment or neurosurgery.

Thus, according to another aspect of the present invention there is provided a method of treating a CNS disease or disorder.

In any of the methods described herein the cells may be obtained from any autologous or non-autologous (i.e., allogeneic or xenogeneic) human donor. For example, cells may be isolated from a human cadaver or a donor subject.

The cells of the present invention can be administered to the treated individual using a variety of transplantation approaches, the nature of which depends on the site of implantation.

The term or phrase “transplantation”, “cell replacement” or “grafting” are used interchangeably herein and refer to the introduction of the cells of the present invention to target tissue. The cells can be derived from the recipient or from an allogeneic or xenogeneic donor.

The cells can be grafted into the central nervous system or into the ventricular cavities or subdurally onto the surface of a host brain. Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the host brain so as to be opposed to the brain parenchyma at the time of transplantation.

Intraparenchymal transplantation can be effected using two approaches: (i) injection of cells into the host brain parenchyma or (ii) preparing a cavity by surgical means to expose the host brain parenchyma and then depositing the graft into the cavity. Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura. Injections into selected regions of the host brain may be made by drilling a hole and piercing the dura to permit the needle of a microsyringe to be inserted. The microsyringe is preferably mounted in a stereotaxic frame and three dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The cells may also be transplanted to a healthy region of the tissue. In some cases the exact location of the damaged tissue area may be unknown and the cells may be inadvertently transplanted to a healthy region. In other cases, it may be preferable to administer the cells to a healthy region, thereby avoiding any further damage to that region. Whatever the case, following transplantation, the cells preferably migrate to the damaged area.

For transplanting, the cell suspension is drawn up into the syringe and administered to anesthetized transplantation recipients. Multiple injections may be made using this procedure.

The cellular suspension procedure thus permits grafting of the cells to any predetermined site in the brain or spinal cord, is relatively non-traumatic, allows multiple grafting simultaneously in several different sites or the same site using the same cell suspension, and permits mixtures of cells from different anatomical regions. Multiple grafts may consist of a mixture of cell types, and/or a mixture of transgenes inserted into the cells. Preferably from approximately 10⁴ to approximately 10⁸ cells are introduced per graft.

For transplantation into cavities, which may be preferred for spinal cord grafting, tissue is removed from regions close to the external surface of the central nerve system (CNS) to form a transplantation cavity, for example as described by Stenevi et al. (Brain Res. 114:1-20., 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the CNS disorder being treated. Demyelinated MS lesions are distributed across multiple locations throughout the CNS, such that effective treatment of MS may rely more on the migratory ability of the cells to the appropriate target sites.

Since non-autologous cells are likely to induce an immune reaction when administered to the body several approaches have been developed to reduce the likelihood of rejection of non-autologous cells. Furthermore, since diseases such as multiple sclerosis are inflammatory based diseases, the problem of immune reaction is exacerbated. These include either suppressing the recipient's immune system, providing anti-inflammatory treatment and/or encapsulating the non-autologous cells in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J. Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE™), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

In any of the methods described herein, the cells can be administered either per se or, preferably as a part of a pharmaceutical composition that further comprises a pharmaceutically acceptable carrier.

As used herein a “pharmaceutical composition” refers to a preparation of one or more of the chemical conjugates described herein, with other chemical components such as pharmaceutically suitable carriers and excipients. The purpose of a pharmaceutical composition is to facilitate administration of a compound to a subject.

Hereinafter, the term “pharmaceutically acceptable carrier” refers to a carrier or a diluent that does not cause significant irritation to a subject and does not abrogate the biological activity and properties of the administered compound. Examples, without limitations, of carriers are propylene glycol, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

According to a preferred embodiment of the present invention, the pharmaceutical carrier is an aqueous solution of saline.

Techniques for formulation and administration of drugs may be found in “Remington's Pharmaceutical Sciences,” Mack Publishing Co., Easton, Pa., latest edition, which is incorporated herein by reference.

Suitable routes of administration include direct administration into the tissue or organ of interest. Thus, for example the cells may be administered directly into the brain as described hereinabove or directly into the muscle as described in Example 3 hereinbelow.

For any preparation used in the methods of the invention, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. For example, animal models of demyelinating diseases include shiverer (shi/shi, MBP deleted) mouse, MD rats (PLP deficiency), Jimpy mouse (PLP mutation), dog shaking pup (PLP mutation), twitcher mouse (galactosylceramidase defect, as in human Krabbe disease), trembler mouse (PMP-22 deficiency). Virus induced demyelination model comprise use if Theiler's virus and mouse hepatitis virus. Autoimmune EAE is a possible model for multiple sclerosis.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1). For example, a multiple sclerosis patient can be monitored symptomatically for improved motor functions indicating positive response to treatment.

For injection, the active ingredients of the pharmaceutical composition may be formulated in aqueous solutions, preferably in physiologically compatible buffers such as Hank's solution, Ringer's solution, or physiological salt buffer.

Dosage amount and interval may be adjusted individually to levels of the active ingredient which are sufficient to effectively regulate the neurotransmitter synthesis by the implanted cells. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. For example, a treated multiple sclerosis patient will be administered with an amount of cells which is sufficient to alleviate the symptoms of the disease, based on the monitoring indications.

The cells of the present invention may be co-administered with therapeutic agents useful in treating neurodegenerative disorders, such as gangliosides; antibiotics, neurotransmitters, neurohormones, toxins, neurite promoting molecules; and antimetabolites and precursors of neurotransmitter molecules such as L-DOPA. Additionally, the cells of the present invention may be co-administered with other cells capable of synthesizing a neurotransmitter. Such cells are described in U.S. Pat. Appl. No. 20050265983 to the present inventors. Additionally, the cells of the present invention may be co-administered with other cells capable of myelination—e.g. Schwann cells, such as those described in U.S. Pat. No. 6,989,271.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Methods

Mesenchymal stem cells: All work with cells was performed using sterile equipment, in sterile class II laminar hoods, and all mediums and solutions were filtered through 0.22 μm sterile filters before use.

A. Growth Medium: MSCs were cultured in Growth Medium (10 ml per flask) containing Dulbecco's modified Eagle's medium (DMEM; Biological Industries) supplemented with 15% heat-inactivated (56° C./30 min) fetal calf serum (FCS; Biological industries), 5% heat-inactivated (56° C./30 min) horse serum (HS; Biological industries), MEM-nonessential amino acids ×1 (MEM-NEAA; Biological industries), 0.001% β-mercaptoethanol (Sigma), 2 mM L-glutamine (Biological Industries), 100 μg/ml streptomycin, 100 U/ml penicillin, 12.5 U/ml nystatin (SPN; Biological industries). The cell cultures were maintained at 37° C. in a humidified 5% CO₂ incubator.

B. Cell harvest: When cells reached 70-90% confluence, cultures were harvested with trypsin-EDTA solution B (0.25% trypsin-EDTA, 1:2000 in Puck's saline; Biological Industries) for 5 minutes at 37° C. The trypsin was neutralized by adding 5 ml of growth medium and the liquid was collected and centrifuged at 1500×g, for 10 min. The pellet was diluted and thoroughly pipetted in 1 ml growth medium, and 10 μl were taken for counting.

C. Cell counting: The cells were counted by multiplying the number of cells in four squares of a hemacytometer (Sigma), and multiplying them ×10⁴ to arrive at the number of cells in the 1 ml volume. Appropriate numbers of cells were replated into flasks or plates for continual growth or differentiation experiments.

Characterization of cell surface markers by fluorescence activated cell sorter (FACS) analysis: Human and mouse mononuclear cells were obtained from total bone marrow as described above. MSCs were harvested from the tissue culture flasks after 14-33 days in vitro and centrifuged at 1000×rpm for 10 min at room temperature. The pellet was re-suspended in PBS and distributed into duplicate samples, suspended in 50 μl PBS. The cells were incubated with appropriate antibodies in the dark (see Table 1, hereinbelow for antibody specifics) for 45 min on ice, washed twice in 2 ml flow-buffer (5% FCS, 0.1% sodium-azide in PBS), and centrifuged for 10 min. The cells were resuspended in 0.5 ml PBS and studied by a fluorescence activated cell sorter (FACS) Calibur™ using an argon ion laser, adjusted to an excitation wavelength of 488 nm (Becton Dickinson Immunocytometry System, San Jose, Calif., http://bdbiosciences.com). The data was acquired and analyzed by CELLQuest™ version-3 software (Becton Dickinson, www.bd.com). A minimum of 10,000 events were examined per sample. A non-specific isotype control was included in each experiment, and specific staining was measured from the cross point of the isotype with the specific antibody graph. Each value is the mean±S.E. if more then two independent experiments were involved. Table 1, hereinbelow summarizes the antibodies used for FACS analysis of cell surface markers.

TABLE 1

| Antibody | Isotype | Manufacturer | Dilution |
|---|---|---|---|
| Fluorescein | Mouse IgG1 | eBioscience | 1:10 |
| isothiocyanate (FITC) | | | |
| Mouse IgG1 Isotype | | | |
| control | | | |
| Phycoerythrin (PE) | Mouse IgG1 | eBioscience | 1:10 |
| Mouse IgG1 Isotype | | | |
| control | | | |
| PE-conjugated anti- | Mouse IgG1 | eBioscience | 1:25 |
| human CD29 | | | |
| FITC- conjugated | Mouse IgG2a | MACS | 1:10 |
| anti-human CD45 | | | |
| FITC- conjugated | Mouse IgG2a | MACS | 1:10 |
| anti-human CD34 | | | |
| FITC- conjugated | Mouse IgG2k | Ancell. Co | 1:100 |
| anti-human CD105 | | | |
| PE- conjugated anti- | Mouse IgG1 | Immuno qual prod. | 1:10 |
| human CD19 | | | |
| PE- conjugated anti- | Unknown | Cymbus Biotec | 1:10 |
| human CD44 | | | |
| PE- conjugated anti- | Rat IgG2b | MACS | 1:20 |
| mouse CD90 | | | |
| PE- conjugated anti- | Rat IgG2a, κ | BioLegend | 1:400 |
| mouse CD106 | | | |
| FITC- conjugated | Rat IgG2b, κ | eBioscience | 1:200 |
| anti-mouse CD45 | | | |

Identification of Oligodendrocyte-Gene Transcripts by Quantitative Real-Time PCR Analysis (qRT-PCR):

A. Preparation and isolation of RNA: Total RNA was extracted from undifferentiated hMSCs, and from hMSCs incubated in the different differentiation mediums by using the mini ribonucleic acid (RNA) I extraction kit (R1006, Zymo, www.zymoresearch.com), following the manufacturer's instructions. Briefly, the cells were scraped off, centrifuged at 400×g for 5 minutes, and following removal of the remaining liquid, RNA extraction buffer was added for 20 minutes, on ice, with light vortexing once every 10 minutes. One volume of 95-100% ethanol was then added and maintained for 10 minutes on ice. The solution was then transferred to zymo spin columns inside collection tubes and centrifuged at 10,000 rpm for one minute, and fluid was discarded. This was repeated, and then 10 μl RNase-free H₂O was added directly to elute the RNA into a clean tube, and following 2 minutes the tubes were centrifuged at 10,000 rpm for one minute.

B. DNase treatment of the RNA samples: 10 μl RNA sample were added to 2.5 μl 10× DNAse buffer, 1.75 μl RNAse free DNAse and 11.25 μl RNAse free water. The mixture was incubated at 37° C. for 15 minutes, and then 4 volumes of RNA binding buffer were added, the solution was transferred to clean zymo spin columns followed by centrifugation at 10,000 rpm for 30 seconds. The upper liquid was discarded and 200 μl RNA wash buffer was added, followed by centrifugation at 10,000 rpm for 45 seconds. This step was repeated, and then 10 μl RNase free H₂O were added and the RNA was eluted by centrifugation at 10,000 for 45 seconds.

The concentration and purity of the RNA was examined by spectrophotometer (Biometra Tgradient, www.biometra.de).

C. Reverse transcription: Reverse transcription was carried out on 0.05 μg/μl messenger RNA (mRNA) samples using the 5 U/μl enzyme SuperScript™ II Ribonuclease (RNase) H⁻Reverse Transcriptase in a mixture containing 2 μM random primers (mostly hexamers), 10 mM dithiotheitol (DTT), 1× buffer supplied by the manufacturer (Invitrogen Life Technologies, www.invitrogen.com), 20 μM dNTPs (TaKaRa Bio Europe, http://www.takarabioeurope.com), and 1 U/μl RNase inhibitor (RNAguard, Amersham Biosciences, www.amershambiosciences.com). The reverse transcription reaction was performed at 25° C. for 10 min, 42° C. for 120 min, 70° C. for 15 min, and 95° C. for 5 min.

D. Quantitative real time PCR: PCR amplifications were performed in a 20 μl final volume containing 1 μl of reverse-transcribed RNA (cDNA), 0.5 μM of sense and anti-sense primers (Agentek), 1× buffer supplied by the manufacturer, 225 μM dNTPs, Taq DNA polymerase 1 unit (TaKaRa), and ddH₂O. Primers for the MBP gene and the 18S housekeeping gene were chosen from different exons to ensure that the PCR products represent the specific mRNA species and not genomic DNA. Primers for the Olig1 gene were from the same exon, hence the importance of the DNase treatment. PCR conditions were optimized by varying the cycle numbers to determine a linear amplification range. The cDNA underwent up to 35 cycles of amplification (1 min at 94° C., 1 min at 54-65° C. and 1 min at 72° C.) in PCR set PTC-100™ (MJ Research, www.mjr.com). The PCR reaction was resolved on a 1% agarose gel. The bands were observed under ultraviolet light and photographed (VersaDoc™ model 1000 Imaging System, Bio-Rad).

### Example 1

### Characterization of MSCs

Results

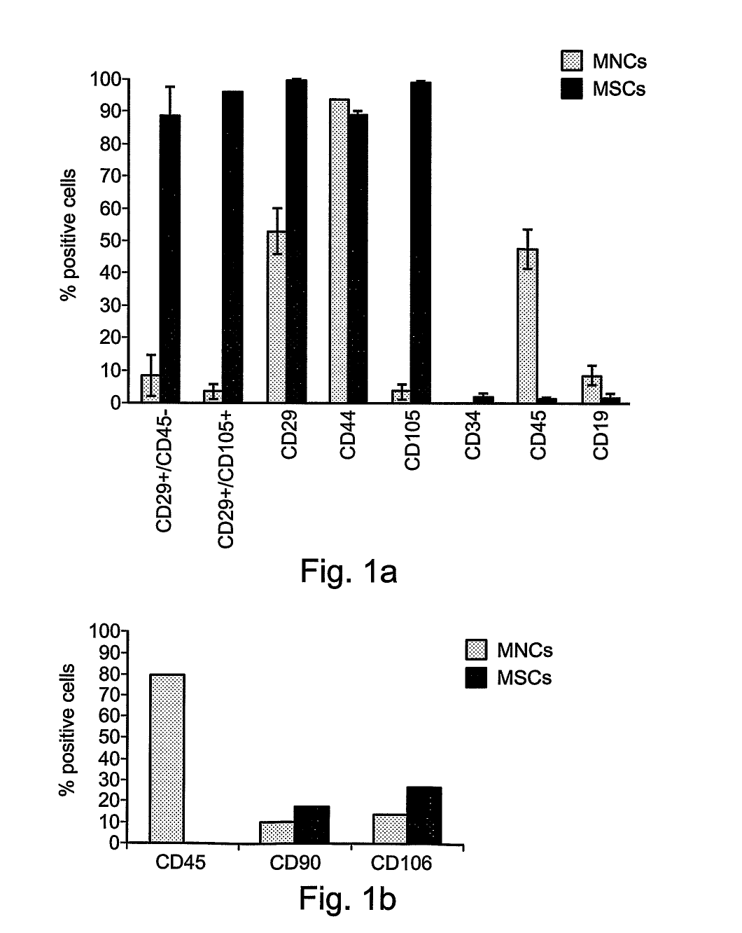

Bone marrow derived plastic adherent cells were characterized by flow cytometry (FACS), and their cell surface marker expression profile was compared with that of total bone marrow derived mononuclear cell (MNC) populations isolated by centrifugation through a density gradient (FIGS. 1A-B).

Briefly, following at least two weeks in vitro the majority of the plastic-adherent population consisted of cells presenting a stable profile of typical mesenchymal surface markers CD105⁺, CD29⁺ and CD44⁺ and a definite majority of the cells were negative for CD34⁻, CD45⁻ and CD19⁻, which are typically negative in MSCs, and characterize other cell types such as hematopoietic cells. Double staining with the mesenchymal CD29 and hematopoietic CD45 or with the mesenchymal CD29 and CD105, were performed as well, and over 88% of the plastic adherent cells showed a mesenchymal pattern of surface marker expression (i.e. were negative for the hematopoietic CD45 marker, but positive for the mesenchymal CD29) with over 90% simultaneously expressing both mesenchymal markers.

The MNC population, on the other hand, displayed a cell marker profile of a mixed cell population, with low levels of mesenchymal markers and relatively high levels of hematopoietic markers. Thus, we concluded that the incubation in vitro enriched for a relatively pure population of plastic adherent mesenchymal cells.

Characterization of mouse bone marrow derived plastic-adherent cells is more complicated, since murine MSCs from different strains vary in the expression of cell-surface markers (Peister A., et al., Blood. 103, 1662-1668, 2004). There are no accepted cell-surface markers for characterization of MSCs from C3H.SW mice, and therefore the expression of two cell-surface markers that are characteristic of human MSCs were examined (FIG. 1): Expression of CD106 was found by Peister A. et al. 2004 (Peister A., et al., Blood. 103, 1662-1668, 2004) to be expressed by less than 20% of mMSCs from BALB/c and DBA1 mice, while up to 75% of MSCs from FVB/n or B1/6 mice expressed this marker. The present inventors found that 27% of the mMSCs from C3H.SW mice expressed CD106. Expression of CD90 was also examined, and it was found that 17% of the mMSCs from C3H.SW mice expressed this marker (whereas Peister A. et al. 2004 found that none of the four strains examined in their study expressed this marker). Importantly, cultured mMSCs from C3H.SW mice display the above typical mesenchymal surface markers at higher levels than freshly isolated MNC populations.

The mMSCs cultures were also examined for the expression of CD45, a characteristic hematopoietic marker. It was found that the cultured cells were depleted of the hematopoietic markers (0% CD45+ cells compared to 80% CD45+ in the mononuclear population). This is especially important because in contrast to human and rat MSCs, the cultures of murine MSCs are frequently contaminated by hematopoietic progenitors that overgrow the cultures.

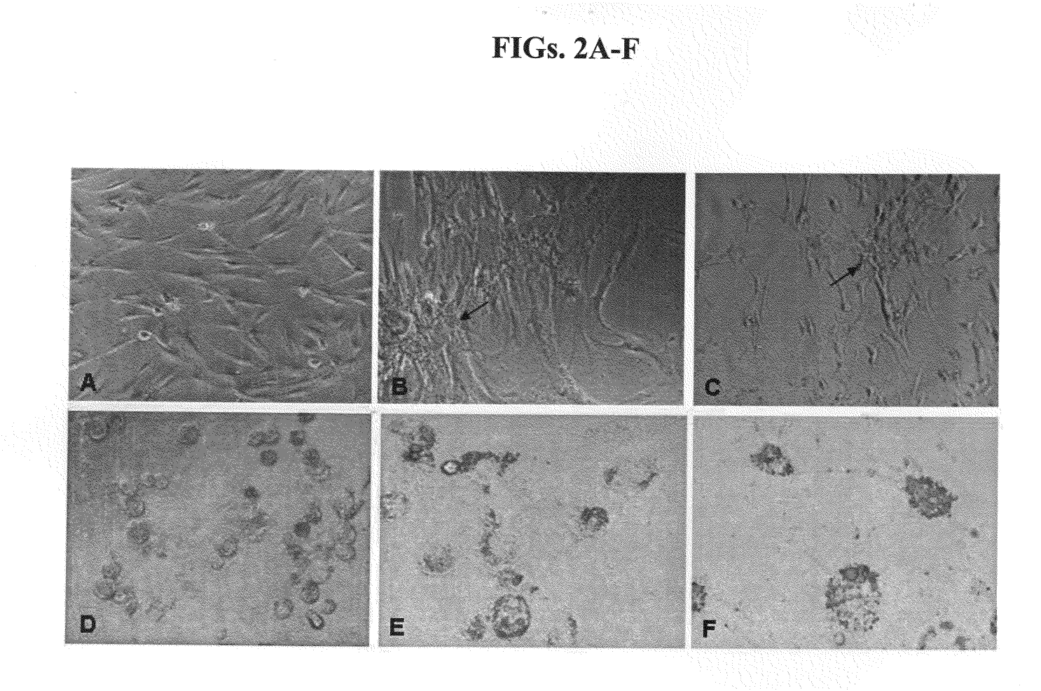

Both mouse and human cultured cells displayed further traits of MSCs, including plastic adherence, typical spindle-like cell morphology and formation of single-cell derived colonies (FIG. 2 A-C).

Moreover, following induction in vitro, by incubation in appropriate specific media, the cells differentiated into fat producing adipocytes (FIGS. 2D-F) and mineral-producing osteoblasts (see Methods, results not shown) thus exhibiting multipotent characteristics.

Thus, it may be concluded that the morphology, clonality, differentiation potential and membrane markers indicate a mesenchymal stem cell identity of the mouse and human cultured cell populations.

### Example 2

### Differentiation of Mouse MSCs to Oligodendrocyte-Like Cells

Materials and Methods

Isolation of mouse MSCs: Mice were sacrificed in a CO₂ chamber and the skin was cleaned in the area of the incisions (hips and legs) using 70% ethanol solution. Sterile scissors were used to isolate the tibias and femurs, and remove muscles and blood vessels. The isolated tibias and femurs were placed in HBSS, and the marrow was removed by insertion of a sterile syringe (1 mL) with a 25-gage needle filled with 0.5 mL sterile HBSS into the bone marrow and flushing out the marrow. Cells were disaggregated by gentle pipetting several times until a milky homogenous single-cell suspension was achieved. Bone marrow aspirates were diluted and washed by adding 5 ml fresh HBSS and centrifugation at 1000×g, for 20 min at room temperature (RT). The supernatant was removed, and the cell pellet was re-suspended in 1 ml growth medium (see below in Cell Culture Conditions) and diluted to 10 ml. The cells were plated in polystyrene plastic tissue-culture 75-cm² flasks (Corning, www.corning.com) and incubated in a humid 37° C. incubator with 5% CO₂. Non-adherent cells were removed following 48 hours. The plastic-adherent cells were considered to be mouse mesenchymal stem cells (mMSCs), as confirmed by subsequent testing. Medium was replaced every 3-4 days.

Oligodendrocyte-like Differentiation of mouse MSCs: 3.2*10³ mMSCs per well from transgenic EGFP+ C57/b1 mice (that have constitutive expression of EGFP in all their cells), were cultured in growth medium (15% FCS, 5% HS, 2 mM L-glutamine, 0.001% β-mercaptoethanol, 10 ng/ml epidermal growth factor, 100 U/ml Penicillin, 100 μg/ml Streptomycin and 12.5 U/ml Nystatin in α-DMEM), and then transferred to mediums containing different cytokines, hormones and growth factors (See table 2 hereinbelow) that play roles in oligodendrocyte lineage differentiation process, to examine the effects of those substances on the cells. The cells were incubated in the differentiation mediums for 48 hours or 6 days, and then were fixed in 4% PFA for 5 minutes in RT and 20 minutes in 4° C. and photographed for morphological changes. Table 2 below summarizes the induction of mMSC differentiation to oligodendrocyte-like cells.

TABLE 2

| | | |
|---|---|---|
| Supplement | Quantity | Days |

| | | | |
|---|---|---|---|
| Interleukin-1β | 20 | ng/ml | 2 or 6 |
| | 40 | ng/ml | |
| db-cAMP | 1 | mM | 2 or 6 |
| | 2 | mM | |
| Retinoic acid | 0.5 | μM | 2 or 6 |
| | 1 | μM | |
| Neurotrophin-3 | 50 | ng/ml | 2 or 6 |
| | 100 | ng/ml | |
| Interleukin-1β | 20 | ng/ml | 2 or 6 |
| Neurotrophin-3 | 50 | ng/ml | |

Manufacturers: db-cAMP, RA from Sigma; NT-3, IL-1β from PeproTech (www.peprotech.com).

The fixed cells were then blocked in 5% goat serum (GS) stained with mouse anti-A2B5 antibodies (1:200; Chemicon) overnight, 4° C., washed twice in 5% GS, and appropriate goat anti mouse second Cy3 antibody (1:500; Jackson labs) for one hour, RT, in the dark. The cells were then examined for fluorescence and photographed by fluorescence Olympus IX70-S8F2 microscope with fluorescent light source and a U-MNU filter cube (Olympus). In all immuno-staining experiments, a sample was also stained with IInd antibodies only, as control, to detect any non-specific staining.

Brain white matter primary culture: As positive control for immunocytochemistry and FACS analysis, brain white matter primary cultures were prepared from the brains of three sacrificed 2-day old mouse pups. After gently removing the bones and exposing the brains, the cerebellums were removed, and the cerebral cortexes were isolated. The cortexes were then placed in Leibovitz-15 medium (Beit Haemek; supplemented by 2 mM L-Glutamine, 15% FCS, 100 U/ml Penicillin, 100 μg/ml Streptomycin and 12.5 U/ml Nystatin), for two washes. The cortexes were then moved to a plate with 2 ml Medium A (1.5 ml DMEM, 0.5 mM EDTA, 0.5 ml trypsin B; Sigma). Using a 1 ml tip, the cortexes were homogenized by gentle pipetting. 4 additional microliters of Medium A were added, and the homogenate was incubated at 37° C. for 10 minutes. Following this, 2 ml Trypsin were added, and the homogenate was shortly pipetted. 15 ml Trituration Medium were added (15 ml DMEM, 30 μl DNase; Sigma, 300 μl Trypsin inhibitor; Sigma). The solution was centrifuged 3 times for 5 minutes at 1200 rpm, and the liquid was discarded. The pellet was diluted in 30 ml DGA Medium (45 ml DMEM, 5 ml FCS, 0.5 ml Glutamine, 0.05 ml Penicillin/Streptomycin/Nystatin). The cells were placed in flasks, and cultured for 24 hours in DGA Medium. Fresh medium was changed every 48 hours, and after a few days the cells were fixed by 4% PFA for 20 min 4° C., and used as positive controls for staining with antibodies.

FACS analysis of differentiated mouse cells: MSCs were harvested from the tissue culture flasks following 7 days in differentiation medium (See Table 3, in the Results, #4), alongside undifferentiated MSCs, and centrifuged at 1000×rpm for 10 minutes at room temperature. The pellet was re-suspended in 200 μl flow buffer (5% FCS, 0.1% sodium-azide in PBS) and distributed into duplicate samples (approximately 1.5*10⁵ cells/sample). The cells were incubated with anti mouse A2B5 antibodies (0.01 mg; Chemicone), for 40 min RT, washed twice in 0.5 ml flow-buffer, and centrifuged for 10 min at 1000 rpm. The cells were resuspended in 200 μl flow buffer and stained with anti mouse Cy3 IInd antibody (1:1000; Chemicon) for 30 min, RT, in the dark. After two washes in flow buffer, the cells were resuspended in 0.5 ml PBS and were studied by a fluorescence activated cell sorter (FACS) Calibur™ using an argon ion laser, adjusted to an excitation wavelength of 488 nm (Becton Dickinson). The data was acquired and analyzed by CELLQuest™ version-3 software (Becton Dickinson). A minimum of 10,000 events were examined per sample. In all immuno-staining experiments, a sample was also stained with 2nd antibodies only, as control, to detect any non-specific staining.

Results

To examine the potential of mouse MSCs to differentiate to oligodendrocyte-like cells, a series of experiments designed to induce the acquirement of oligodendrocyte phenotype was performed (see Table 3, hereinbelow). Based on an extensive review of the literature, different combinations of growth factors and cytokines were added to serum-free medium in which mouse MSCs were cultured for different amounts of time, and then examined for changes in morphology and gene expression. Table 3 below summarizes the differentiation experiments to oligodendrocyte-like cells.

TABLE 3

| Substances | Cells | Duration | Detection | Results |
|---|---|---|---|---|
| Added to Growth | 3*10⁴ EGFP | 2 days | Morphology | Few A2B5+ cells |
| Medium, in 19 | mMSCs per | | Immuno- | in bFGF +dbCAMP |
| different | well | | staining: A2B5 | treatment. |
| combinations: | | | | |
| EGF (10 ng/ml) | | | | |
| bFGF(10 ng/ml) | | | | |
| IL-1β (20 or 40 | | | | |
| ng/ml) | | | | |
| dbcAMP (1 or 2 | | | | |
| mM) | | | | |
| RA (0.5 or 1 μM) | | | | |
| NT-3 (50 or 100 | | | | |
| ng/ml) | | | | |
| Added to Growth | 3.2*10³ C57- | 2 or 6 days | Morphology | A2B5+ cells |
| Medium, in 15 | EGFP mMSCs | | Immuno- | detected at up to |
| different | per well | | staining: A2B5 | 8% in some of the |
| combinations: | | | | treatments. See |
| EGF (10 ng/ml) | | | | Results section. |
| bFGF(10 ng/ml) | | | | |
| IL-1β (20 or 40 | | | | |
| ng/ml) | | | | |
| dbcAMP (1 or 2 | | | | |
| mM) | | | | |
| RA (0.5 or 1 μM) | | | | |
| NT-3 (50 or 100 | | | | |
| ng/ml) | | | | |
| Added to Growth | 2.4*10⁵ EGFP | 4 days | Morphology | Oligodendrocyte- |
| Medium: | mMSCs per | | FACS analysis: | like |
| IL-1β 20 ng/ml | plate | | A2B5 | Morphological |
| | | | | changes. Low |
| | | | | percentage |
| | | | | A2B5+ cells |
| | | | | following |
| | | | | differentiation. |
| Added to Growth | 6*10³ cells per | 7 days | Morphology | No significant |
| Medium: | well | | FACS analysis: | staining detected |
| IL-1β (20 ng/ml) | 2*10⁶ cells per | | A2B5, O4 | by FACS (also |
| NT-3 (50 ng/ml) | plate | | Immuno- | not in positive |
| Or: | C57b1 mMSCs | | staining: A2B5 | control). |
| TH (30 ng/ml) | or Brain cells | | | |
| PDGF (20 ng/ml) | from EGFP+ | | | |
| bFGF (10 ng/ml) | one day old | | | |
| Glial primary | mouse as | | | |
| culture | positive control | | | |
| supernatant | | | | |
| (1:10; see | | | | |
| Methods) | | | | |
| Added to Growth | 1.5*10⁴ EGFP | 6 days | Morphology | Oligodendrocyte- |
| Medium: β | mMSCs per 48- | | Immuno | like |
| mercaptoethanol | well | | staining: | Morphological |
| (1 mM) | | | A2B5, O4 | changes. No |
| bFGF (10 ng/ml) | | | | significant |
| PDGF (20 ng/ml) | | | | staining detected |
| TH (30 ng/ml) | | | | |
| N2 medium (X1) | | | | |
| IL-1β (20 ng/ml) | | | | |
| NT-3 (50 ng/ml) | | | | |
| Added to Growth | 2*10³ CNP- | 14 days | Morphology | Detection of |
| Medium: | C3H mMSCs | | ELISA readings | EGFP+ level not |
| Shh (200 ng/ml) | per 96-well | | for detection of | achieved. No |
| TH (30 ng/ml) | | | elevations in | significant |
| IL-1β (40 ng/ml) | | | EGFP levels | staining. No |
| NT-3 (100 ng/ml) | | | (i.e. CNP | significant |
| PDGF (20 ng/ml) | | | promotor | morphological |
| BDNF (10 ng/ml) | | | activation) | change. |
| | | | Immuno | |
| | | | staining: | |
| | | | A2B5, MBP | |

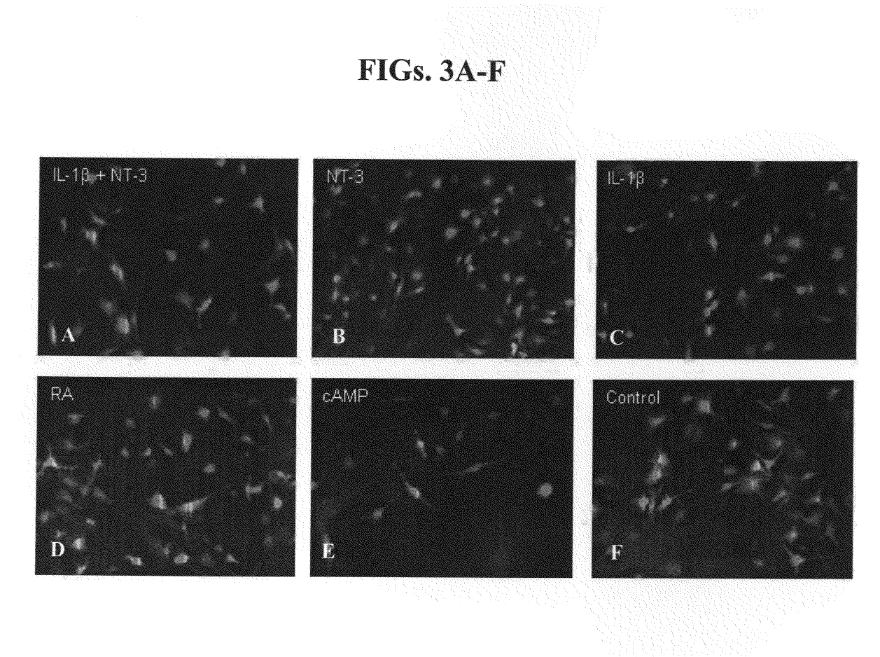

It was found that up to 8% of the cells expressed the oligodendrocyte precursor marker A2B5, following treatment with IL-1β and NT-3 for 8 days (FIGS. 3A-F and 4A-C). Significant morphological changes were not detected in the cultured A2B5+ cells, however, this could be due to the similarity in the morphologies of oligodendrocytes precursors and MSCs.

Table 4, hereinbelow summarizes the percent of mouse MSCs induced to express A2B5 using different protocols.

TABLE 4

| | |
|---|---|
| Cytokines added to growth | Fraction of A2B5+ cells |

| medium: | 48 hours | 6 days |
|---|---|---|
| Medium only | 0 | 0 |
| Interleukin-1β | 1% | 3.8% |
| Neurotrophin-3 | 1.9% | 2.7% |
| IL-1β + NT-3 | — | 8% |
| Retinoic acid | 0% | — |
| cAMP | 0.6% | — |

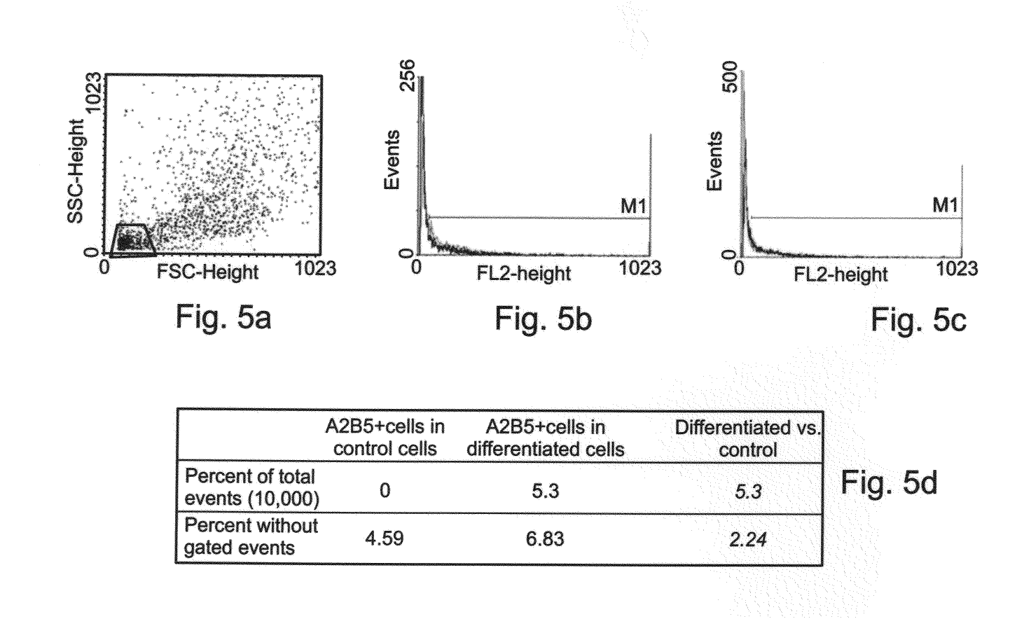

An attempt to characterize the exact numbers of A2B5+ cells in a cell population incubated in medium supplemented by IL-1β using FACS analysis (FIGS. 5A-D) detected similar numbers, strengthening these ‘roughly estimated’ counts. The use of FACS analysis for detection of such low numbers lies on the borderline of the machine's detection abilities.

### Example 3

### Differentiation of Human MSCs to Oligodendrocyte-Like Cells

Materials and Methods

Isolation of human MSCs: The study was approved by the Helsinki ethical committee of the Israeli Ministry of Health and Tel-Aviv University, and individual informed consent was obtained from donors. Bone marrow aspirates (10 ml) were obtained from iliac crests of human donors (age range 19-76 years old). No significant differences between the samples were detected. The aspirates were diluted 1:1 in 10 ml of Hank's balanced salt solution (HBSS; Biological Industries http://www.bioind.com). Using a Pasteur pipette a quarter volume Ficoll solution (1.077 g/ml) was added underneath the bone marrow sample. Mononuclear cells were isolated by centrifugation at 2500×g for 30 min at room temperature through the Ficoll density gradient (Histopaque®-1077; Sigma). The mononuclear cell layer was recovered from the gradient interface by Pasteur pipette, washed with HBSS and centrifuged at 2000×g for 20 min at room temperature. The cells were re-suspended in Growth Medium (see below in Cell Culture Conditions), plated in polystyrene plastic 75-cm² tissue-culture flasks (Corning, N.Y., http://www.corning.com) and incubated at 37° C. humid incubator with 5% CO₂. Non-adherent cells were removed following 48 hours. The plastic-adherent cells were considered to be human mesenchymal stem cells (hMSCs), as confirmed by subsequent testing. Medium was replaced every 3-4 days.

Oligodendrocyte-like Differentiation of human MSCs: 2*10⁵ hMSCs per plate, from two donors (serving as biological duplicates) were cultured for at least 14 days in growth medium, as described above, and then they were transferred to mediums containing different cytokines, hormones and growth factors that play roles in oligodendrocyte maturation and lineage differentiation process, to examine the effects of those substances on the cells. The serum-free protocols that were used are described in Table 5 hereinbelow, most of which consisted of a stage consisting of an assortment of growth factors, followed by a growth-factor withdrawal stage, including cytokines intended to ‘push’ the cells to differentiate. The cells were incubated in the differentiation mediums for times indicated (a total of 13 days), and were photographed every two-three days for detection of morphological changes. Table 5 below summarizes the induction of hMSC differentiation to oligodendrocyte-like cells.

TABLE 5

| | | |
|---|---|---|
| Stage | Medium^{a} | Days |

Control

| | |
|---|---|
| Regular growth medium: | 13 |
| α-MEM | |
| FCS 15% | |
| L-glutamine 2 mM | |
| Penicillin 100 U/ml | |
| Streptomycin 100 ug/ml | |
| Nystatin 12.5 U/ml | |

Protocol A

| | | |
|---|---|---|
| Differentiating | Neurobasal medium | 13 |
| Medium (A) | N2 supplement | |
| | B27 supplement | |
| | bFGF 10 ng/ml | |
| | GGF 50 ng/ml | |
| | db-cAMP 1 nM | |

Protocol B

| | | |
|---|---|---|
| Additional Medium (B) | Neurobasal medium | 5 |
| | PDGF 20 ng/ml | |
| | NT-3 10 ng/ml | |
| | I1-1β 20 ng/ml | |
| Differentiating | Neurobasal medium | 8 |
| Medium (B) | N2 supplement | |
| | NT-3 10 ng/ml | |
| | I1-1β 20 ng/ml | |

Protocol C

| | | |
|---|---|---|
| Additional Medium (C) | Neurobasal medium | 5 |
| | TH 30 ng/ml (stock 20 | |
| | ug/ml) | |
| | RA 1 μM | |
| | GGF 50 ng/ml | |
| Differentiating | Neurobasal medium | 8 |
| Medium (C) | TH 30 ng/ml (stock 20 | |
| | ug/ml) | |
| | RA 1 μM | |
| | NT-3 10 ng/ml | |

Protocol D

| | | |
|---|---|---|
| Additional Medium (D) | Neurobasal medium | 5 |
| | PDGF 20 ng/ml | |
| | GGF 50 ng/ml | |
| Differentiating | Neurobasal medium | 8 |
| Medium (D) | Shh 300 ng/ml | |
| | NT-3 10 ng/ml | |
| | db-cAMP 1 nM | |
| | Forskolin 5 μM | |

^{a}L-Glutamine (2 mM) and SPN antibiotics (Pen: 100 U/ml Strep: 100 ug/ml Nyst: 12.5 U/ml) were added routinely to all treatments.
^{b}N2 supplement: 5 μg/ml insulin; 20 nM progesterone; 100 μM putrescine; 30 nM selenium; 100 μg/ml transferrin.
Abbreviations: FCS, fetal calf serum; bFGF, basic fibroblast growth factor; GGF, glial growth factor; db-cAMP, dibutyryl cyclic AMP; PDGF, platelet derived growth factor; NT-3, neurotrophin 3; IL-1β, interleukin 1 beta; TH, thyroid hormone; RA, retinoic acid; Shh, sonic hedgehog.
Manufacturers: bFGF, Shh, GGF, PDGF, are from R&D (www.rndsystems.com); db-cAMP, RA, TH and N2 supplements are from Sigma; NT-3, IL-1β are from PeproTech.

Immunostaining of the differentiated human cells: At the end of the differentiation period, the cells were stained for expression of oligodendrocyte specific markers. The fixed cells were then blocked in 5% GS, stained with anti-A2B5 antibodies (hybridoma in DMEM; gift of Michal Geva), or anti-MOSP antibodies (1:50; Chemicon) for 30 minutes, RT. The cells were then washed twice in 5% GS (A2B5-stained cells were washed in DMEM), and were fixed in 2% PFA, RT and transferred to 5% GS overnight, 4° C. A solution containing Hoechst 33342 (1:1000, Sigma) nuclear dye and Cy3-conjugated anti IgM antibody (1:500; Chemicon) in 5% GS were added on the cells for 40 minutes, RT, followed by 2 washes in 5% GS and one wash in PBS. The cells were then examined for fluorescence and photographed by the fluorescence Olympus IX70-S8F2 microscope with fluorescent light source and a U-MNU filter cube (Olympus). In all immuno-staining experiments, a sample was also stained with IInd antibodies only, as control, to detect any non-specific staining.

Results

To examine the potential of human MSCs to differentiate to oligodendrocyte-like cells, a series of experiments designed to induce the acquirement of oligodendrocyte phenotype was performed (see Table 6, hereinbelow). Based on an extensive review of the literature, different combinations of growth factors and cytokines were added to serum-free medium in which human MSCs were cultured for different amounts of time, and then examined for changes in morphology and gene expression. Table 6 below summarizes the differentiation experiments to oligodendrocyte-like cells.

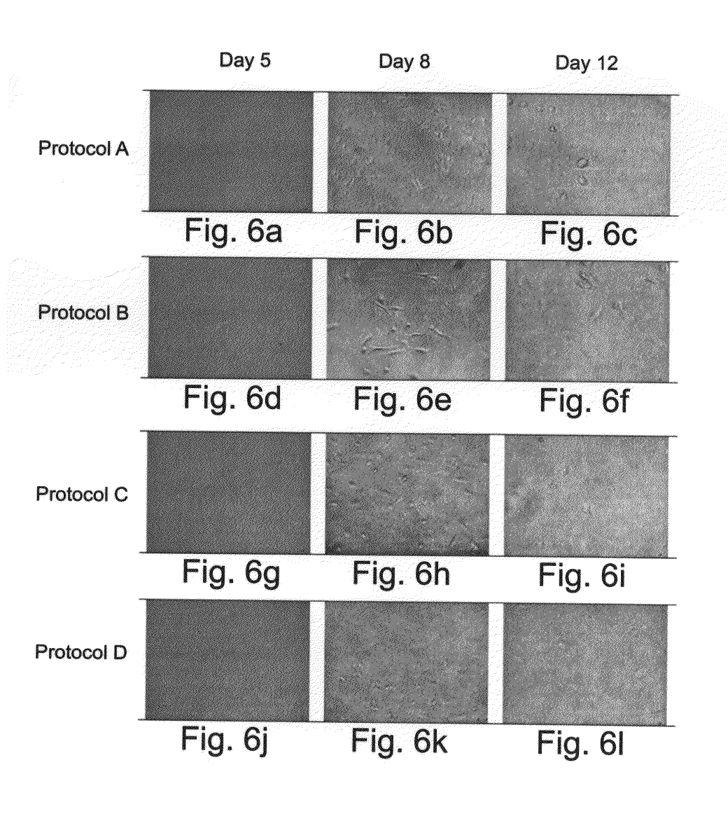

TABLE 6

| Substances | Cells | Duration | Detection | Results |
|---|---|---|---|---|
| DMEM | 6*10³ hMCSs | 6, 10 or 14 | Morphology | Some |
| 10% FBS | per well | days | Immuno | morphological |
| RA (35 ng/ml) | | | staining: | changes were |
| bFGF (10 ng/ml) | | | A2B5, MOSP, | detected, however |
| PDGF (5 ng/ml) | | | MBP | no significant |
| GGF (50 ng/ml) | | | | staining was |
| Or: | | | | found. |
| In growth | | | | |
| medium | | | | |
| N2 supplement | | | | |
| bFGF (10 ng/ml) | | | | |
| PDGF (5 ng/ml) | | | | |
| In growth | hMSCs donor | 7 days | Morphology | Marked |
| medium: | #100 | | Western blot: | morphological |
| N2 supplement | | | MBP | changes to |
| Shh (300 ng/ml) | | | Real-time PCR | oligodendrocyte - |
| Forskolin (5 μM) | | | for Olig1 and | like (multi- |
| Or: N2 medium | | | MBP mRNA | procceses) cells. |
| GGF (50 ng/ml) | | | transcripts | DNA traces in the |
| cAMP (1 nM) | | | | mRNA samples, |
| bFGF (10 ng/ml) | | | | and faulty DNAse |
| Or: IL-1β (20 | | | | treatment of the |
| ng/ml) | | | | samples skewed |
| NT-3 (10 ng/ml) | | | | results. |
| PDGF (20 ng/ml) | | | | No staining for |
| | | | | MBP was found |
| | | | | in the blots. |
| In Neurobasal | 2*10⁵ hMSCs | 12 days | Morphology | Cells developed |
| medium 5 | per plate, | | Immuno | multiple process |
| assortments of | donors #104 | | staining: A2B5, | and assumed |
| the following: | and 107 | | MOSP | oligodendrocyte- |
| N2 supplement | (biological | | Real-time PCR: | like morphology |
| bFGF 10 ng/ml | duplicates) | | MBP, Olig1 | (FIG. 6-10). |
| PDGF 20 ng/ml | | | | Elevated MBP |
| IL-6 chimera X1 | | | | mRNA in some |
| GGF 50 ng/ml | | | | of the treatments, |
| cAMP 1 nM | | | | cells positive for |
| B27 supplement | | | | A2B5 and MOSP |
| NT-3 10 ng/ml | | | | in some of the |
| IL-1β 20 ng/ml | | | | treatments. |
| T-3 30 ng/ml | | | | |
| RA 1 μM | | | | |
| Shh 300 ng/ml | | | | |
| Forskolin 5 μM | | | | |

An important difference between the differentiations protocols used for mouse vs. human MSCs was that the mouse cells were grown with serum throughout the differentiation process, whereas the human MSCs were cultured in serum-free conditions.

Table 7 hereinbelow summarizes the differentiation experiments to oligodendrocyte-like cells using protocols A-D.

TABLE 7

| | Oligodendrocyte -like | Elevation of MBP | Oligodendrocyte |
|---|---|---|---|
| Protocol | morphology | mRNA | markers examined |
| Control | − | − | − |
| Protocol A | + | + | − |
| Protocol B | ++ | ++ | ++ |
| Protocol C | + | ++++ | − |
| Protocol D | +++ | + | ++ |

Differentiation experiments in serum-free conditions with the human MSCs, resulted in marked morphological changes following incubation in the diverse differentiation mediums (FIGS. 6A-O and Table 7 hereinabove). While morphological change is not, in itself, enough to define differentiation, it does factor in the bigger picture.

The expression of MBP, a key gene in oligodendrocyte maturation and myelin-production, was examined in order to assess the differentiation of the MSCs, following administration of the different differentiation protocols. As MSCs are known to express a wide variety of neural genes (Blondheim N. R., et al., Stem Cells Dev. 15, 141-164, 2006; Deng J., et al., Stem Cells. 24, 1054-1064, 2006), real-time PCR was used to quantify the levels of MBP transcripts in the cells. Undifferentiated human MSCs did express MBP mRNA at a low basal level, however, following incubation in the differentiation mediums the level of MBP mRNA increased dramatically (Table 8). Table 8 hereinbelow summarizes the relative quantification using the comparative CT method.

TABLE 8

| | | | | | MBP |
|---|---|---|---|---|---|
| | MBP | 18S | ΔC_{T} | ΔΔC_{T} | relative to |
| Treatment^{a} | average C_{T} | average C_{T} | MBP − 18S^{b} | ΔC_{T} − ΔC_{T(C)}^{c} | control A^{d} |
| Control₁ | 39.65 | 23.57 | 16.08 ± 1.87 | 0.00 ± 1.87 | 1.00 |
| | | | | | (0.72-1.27) |
| Control₂ | 38.93 | 22.72 | 16.21 ± 0.73 | 1 ± 0.73 | 1.00 |
| | | | | | (0.39-1.60) |
| Protocol A₁ | 40.95 | 26.37 | 14.58 ± 0.69 | −1.50 ± 0.69 | 2.83 |
| | | | | | (1.08-4.59) |
| Protocol A₂ | 40.87 | 26.18 | 14.70 ± 0.94 | −1.51 ± 0.94 | 2.85 |
| | | | | | (1.36-4.34) |
| Protocol B₁ | 41.60 | 31.20 | 10.40 ± 0.46 | −5.67 ± 0.46 | 51.09 |
| | | | | | (14.06-88.12) |
| Protocol B₂ | — | 27.44 | — | — | — |
| Protocol C₂ | 40.12 | 30.70 | 9.42 ± 1.02 | −6.79 ± 1.02 | 110.92 |
| | | | | | (56.29-165.59) |
| Protocol D₁ | 41.65 | 29.67 | 11.98 | −4.10 | 17.15 |
| Protocol D₂ | 38.18 | 22.42 | 15.77 ± 0.60 | −0.44 ± 0.60 | 1.36 |
| | | | | | (0.46-2.25) |

^{a}1 = hMSCs from donor #104, 2 = hMSCs from donor #107.
^{b}The ΔC_{T} value is determined by subtracting the average 18S ΔC_{T} value from the average MBP ΔC_{T} value. The standard deviation of the difference is calculated from the standard deviations of the MBP and 18S values.
^{c}The calculation of ΔΔC_{T} involves subtraction by the ΔC_{T} calibrator value (in this case, ΔC_{T} of Control sample₁ or ₂, accordingly). This is subtraction of an arbitrary constant, so the standard deviation of ΔΔC_{T} is the same as the standard deviation of the ΔC_{T} value.
^{d}The range given for MBP relative to brain is determined by evaluating the expression: 2^{\hat{ }}ΔΔCT with ΔΔCT + s and ΔΔCT − s, where s = the standard deviation of the ΔΔCT value (calculated as the square root of the sum of s_{I}² + s_{II}²).
Abbreviation: C_{T} = cycle threshold

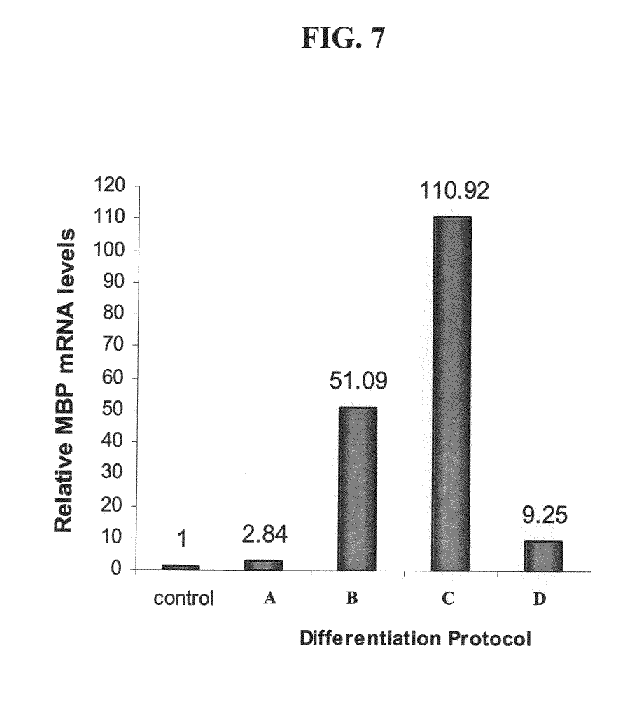

An average of the results of cells from both donors that participated in the experiment is presented in FIG. 7. No increase in Olig1 mRNA was detected by real-time PCR. Notably, differentiation protocols B and D showed elevated levels of the MBP mRNA in some of the treatments. Interestingly, protocols B and D also had induced remarkable increases in the morphological complexity of the cells (FIGS. 8A-G).

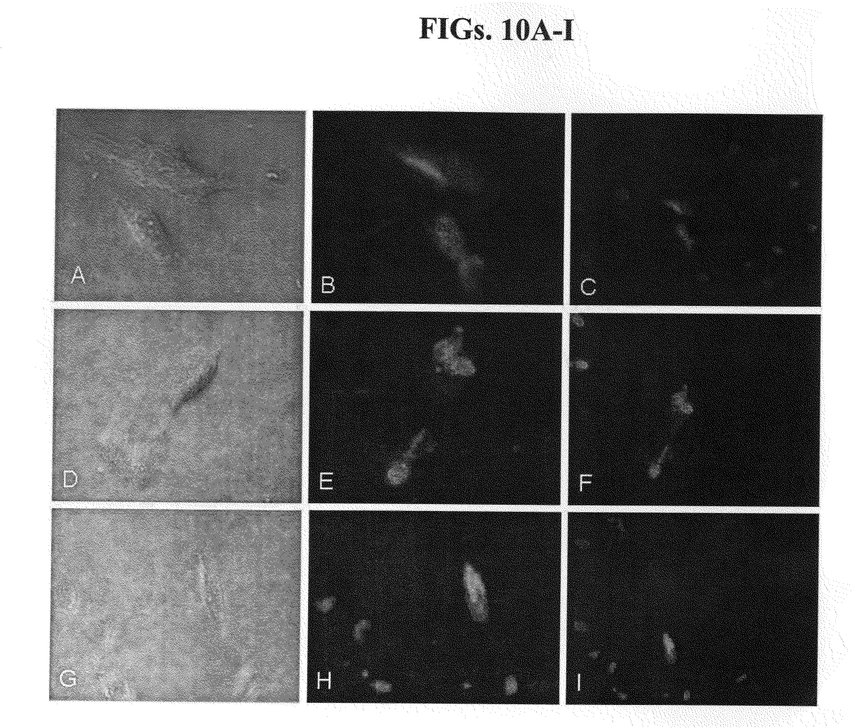

Expression of additional oligodendrocyte markers was examined by immunocytochemistry: A2B5 (FIGS. 9A-C) and MOSP (FIGS. 10A-I) were detected in a portion of the human MSCs following differentiation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. An isolated human cell comprising at least one oligodendrocyte phenotype and at least one mesenchymal stem cell phenotype, wherein said mesenchymal stem cell phenotype is not said oligodendrocyte phenotype.

2-3. (canceled)

4. An isolated cell population comprising human cells wherein:
(i) at least N % of said human cells comprise at least one oligodendrocyte phenotype;
(ii) at least M % of said human cells comprise at least one mesenchymal stem cell phenotype, said mesenchymal stem cell phenotype is not an oligodendrocyte phenotype; and
(iii) at least one of said human cells comprises both said at least one oligodendrocyte phenotype and said at least one mesenchymal stem cell phenotype;
where M and N are each independently selected between 1 and 99.

5-6. (canceled)

7. The isolated human cell of claim 1, being non-genetically manipulated.

8. The isolated human cell of claim 1, wherein said at least one oligodendrocyte phenotype is a structural phenotype.

9. The isolated human cell of claim 1, wherein said at least one oligodendrocyte phenotype is a functional phenotype.

10-14. (canceled)

15. The isolated human cell of claim 8, wherein said oligodendrocyte structural phenotype is expression of at least one oligodendrocyte marker.

16-19. (canceled)

20. A method of generating oligodendrocyte-like cells, comprising incubating mesenchymal stem cells in a differentiating medium comprising NT-3, thereby generating oligodendrocyte-like cells.

21-22. (canceled)

23. The method of claim 20, wherein said medium comprises N2 supplement and bFGF.

24. (canceled)

25. The method of claim 20, wherein a concentration of said NT-3 is about 10 ng/ml.

26. The method of claim 20, wherein said differentiating medium further comprises at least one agent selected from the group consisting of Il-1β, N2 supplement, TH, RA, Shh, db-cAMP and forskolin.

27-29. (canceled)

30. The method of claim 20 further comprising culturing the cells in an additional medium prior to said incubating thereby predisposing said cells to differentiate into oligodendrocyte-like cells.

31. The method of claim 30, wherein said additional medium comprises at least one agent selected from the group consisting of PDGF, NT-3, Il-1β, TH, RA and GGF.

32-35. (canceled)

36. A method of generating oligodendrocyte-like cells, comprising incubating mesenchymal stem cells in a differentiating medium comprising N2 supplement and bFGF, thereby generating oligodendrocyte-like cells.

37. (canceled)

38. The method of claim 36, wherein a concentration of said bFGF is about 10 ng/ml.

39-43. (canceled)

44. A method of treating a medical condition of the CNS, the method comprising administering to a subject in need thereof a therapeutically effective amount of the cell population of claim 4, thereby treating the CNS disease or disorder in the subject.

45-47. (canceled)

48. The method of claim 44, wherein the CNS disease or disorder is multiple sclerosis.

49-51. (canceled)

52. The isolated cell population of claim 4, being non-genetically manipulated.

53. The isolated cell population of claim 4, wherein said at least one oligodendrocyte phenotype is a structural phenotype.

54. The isolated cell population of claim 4, wherein said at least one oligodendrocyte phenotype is a functional phenotype.

55. The isolated cell population of claim 53, wherein said oligodendrocyte structural phenotype is expression of at least one oligodendrocyte marker.

56. The method of claim 23, wherein a concentration of said bFGF is about 10 ng/ml.
